(19) United States
(12) Patent Application Publication
(10) Pub. No.: US 2022/0118166 A1
(43) Pub. Date: Apr. 21, 2022

(54) WOUND CARE DEVICE FOR TREATING WOUNDS BY MEANS OF SUBATMOSPHERIC PRESSURE

(71) Applicant: BSN medical GmbH, Hamburg (DE)

(72) Inventor: Birgit RIESINGER, Munster (DE)

(21) Appl. No.: 17/564,433

(22) Filed: Dec. 29, 2021

Related U.S. Application Data
(63) Continuation of application No. 14/289,195, filed on May 28, 2014, now abandoned.
Application No. 14/289,195 is a continuation of application No. PCT/EP2012/004916, filed on Nov. 28, 2012.

(30) Foreign Application Priority Data
Nov. 28, 2011 (DE) 10 2011 055 782.2

(51) Int. Cl. A61M 1/00

(52) U.S. Cl. CPC A61M 1/90; A61M 2205/42; A61M 2205/8206; A61M 1/962

(57) ABSTRACT

The subject matter of the invention is a wound care device for treatment of wounds by means of subatmospheric pressure in the wound region, having a wound covering element, a device for generating subatmospheric pressure, which can optionally be placed on the wound covering element, and an absorption body taking up wound exudations.

### CROSS-REFERENCE TO RELATED APPLICATIONS

The present application claims priority from and is a continuation of co-pending U.S. application Ser. No. 14/289195, filed May 28, 2014, which is a continuation of PCT Application No. PCT/EP2012/004916, filed Nov. 28, 2012, which claims priority from German Patent Application No. DE 10 2011 055 782.2, filed Nov. 28, 2011, each of which are herein incorporated by reference in their entireties.

### FIELD OF THE INVENTION

The invention concerns a wound care device for treatment of wounds by means of subatmospheric pressure.

### BACKGROUND

Traditional systems and devices for treatment of wounds by means of subatmospheric pressure consist of a gas-tight wound covering, a drainage hose, an externally arranged vacuum pump, and a collecting vessel to take up the drained exudations. Examples of such devices are described in U.S. Pat. No. 7,198,046. The drawback to the systems and devices disclosed in U.S. Pat. No. 7,198,046 is that said devices are generally not designed to be mobile for treatment of wounds by means of subatmospheric pressure. A therapy is only possible locally, for example, through a pump situated at the patient's bed. This ties the patient to the bed restricting the patient's freedom of movement. If the patient needs to move, the pump is removed and the therapy is interrupted.

There is a need in the art for a vacuum wound treatment that is more comfortable to the patient. There is also a need in the art for a continuous vacuum wound treatment that would not require interrupting the therapy when the patient is mobile.

### SUMMARY OF THE INVENTION

In view of the above, a wound care device is provided for treatment of wounds by means of subatmospheric pressure in the wound area. In one aspect of the invention, an example implementation of a wound care device comprises at least one wound covering element, and at least one device for generating subatmospheric pressure. The device for generating subatmospheric pressure may optionally be placed on the wound covering element. The device may include at least one absorption body for absorbing wound exudations. Alternatively or in addition to this, the device may also include at least one reservoir for receiving wound exudations. One example of a reservoir that may be used is a canister. The device for creating subatmospheric pressure may be one of the following:

a) electrically operated vacuum pump

b) manually operated partial vacuum source, and/or

c) evacuated vacuum vessel.

The electrically operated vacuum pump may be a single pump, or a pump that is part of a centralized suction system, such as is often used in clinics. In this case, wall-installed vacuum connections are provided in the patient rooms, to which drainage devices may be connected for the wound treatment. In this case, the vacuum pump may be used to place a number of drainage devices for wound treatment under a partial vacuum.

In an example implementation, the vacuum pump may be a micropump, whose dimensions and/or whose weight is such that it may be easily placed on a wound covering element of the kind described above without being a burden to the patient.

The micropump may be a piezo or membrane pump, for example. Piezo pumps are pumps in which the pump power is produced by a piezoelectric element. These pumps have a sufficiently high pump power with slight dimensions, slight operating noise, and low energy consumption. The vacuum pump may also be a propellant vacuum pump such as those used in microsystem technology. Suitable pumps of this type are, for example, those manufactured by Schwarzer Precision, KNF or Bartels Mikrotechnik.

The vacuum pump may also be outfitted with a check valve. The check valve permits the pump to be operated in interval mode or only turned on for the initial build-up of a partial vacuum or to maintain a partial vacuum, without any leaks occurring in the operating intermissions that may dissipate the partial vacuum built up. p The pump may be configured to draw partial vacuums ranging from −20 to −200 mm Hg. The pump may draw the partial vacuums at levels of −20, −30, −40, −50, −60, −70, −80, −85, −90, −100, −110, −120, −130, −140, −150, −160, −170, −180, −190 or −200 mm Hg. More preferred partial vacuums range from −60 to −110 mm Hg, at levels of −60, −61, −62, −63, −64, −65, −66, −67, −68, −69, −70, −71, −72, −73, −74, −75, −76, −77, −78, −79, −80, −81, −82, −83, −84, −85, −86, −87, −88, −89, −90, −91, −92, −93, −94, −95, −96, −97, −98, −99, −100, −101, −102, −103, −104, −105, −106, −107, −108, −109 and/or −110 mm Hg. It is to be understood that the pressure values disclosed and discussed herein are relative to a normal pressure of 1 bar=750 mm Hg.

In some example implementations, the device includes an absorbing body made of a superabsorbing polymer. In such implementations, there is a synergy between superabsorbing polymers and subatmospheric pressure in which there is both a suction and a mobilizing action on the exudations present in the wound. Implementations using both a superabsorbing polymer and subatmospheric pressure allow for the use of a smaller partial vacuum than is usual in the prior art, since—as described—the subatmospheric pressure is complimented by the superabsorbing polymers. In this way, the operating noise and the associated vibrations can be decreased, the battery size reduced, and/or the battery life extended. The partial pressures used in such implementations may be in the range between −20 and −80 mm Hg, at levels of −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79 and/or −80 mm Hg.

In another example implementation, the partial vacuums are provided in the range between −100 and −140 mm Hg, at levels of −100, −101, −102, −103, −104, −105, −106, −107, −108, −109, −110, −111, −112, −113, −114, −115, −116, −117, −118, −119, −120, −121, −122, −123, −124, −125, −126, −127, −128, −129, −130, −131, −132, −133, −134, −135, −136, −137, −138, −139, and/or −140 mm Hg. In another example embodiment, the partial vacuums are provided in the range between −110 and −135 mm Hg, at levels of −110, −111, −112, −113, −114, −115, −116, −117, −118, −119, −120, −121, −122, −123, −124, −125, −126, −127, −128, −129 and/or −130 mm Hg. In yet another example embodiment, the partial vacuums are provided in the range between −120 and −125 mm Hg, at levels of −120, −121, −122, −123, −124 and/or −120 mm Hg. The pump may also be selected and outfitted such that it is able to deliver liquids.

It is preferred in an example implementation that the operating noise of the pump not exceed a sonic pressure level of 75 dB. Sonic pressure levels of 73 dB, 70 dB, 68 dB, 65 dB, 63 dB, 60 dB, 58 dB, 55 dB, 53 dB, 50 dB, 48 dB, 45 dB, 42 dB, 40 dB, 38 dB, 35 dB, 32 dB, 30 dB, 28 dB, 25 dB 22 dB, or even 20 dB should not be exceeded.

The reduction of the operating noise may also be accomplished by using a pump housing that is encased or lined in a soundproofing material. Foams such as neoprene or the like are examples of soundproofing material that may be used. The housing and/or the interior engineering can be mounted or made from a nonrigid material.

In an example implementation, the pump has a delivery rate between 0.5 ml and 100 ml min⁻¹. The delivery rates may be, for example, at levels of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 ml min⁻¹. Preferred delivery rates range between 2 ml min⁻¹ and 50 ml min⁻¹. More preferred delivery rates range between 10 ml min⁻¹and 20 ml min⁻¹.

In some example implementations, the device for creating subatmospheric pressure includes an evacuated vessel. The evacuated vessel may be connected to the device for wound treatment in a manner similar to the familiar Redon bottle to place the device under partial vacuum. The evacuated vessel may include an insert containing liquid-absorbing polymers, preferably in the form of a wall lining. The evacuated vessel may be provided in the form of a cartridge inserted into a holder that is already connected to a drainage device for wound treatment. When the cartridge is full, it is removed and disposed of, and a new evacuated cartridge can be inserted into the holder.

Example implementations using the evacuated vessel advantageously allow for the device to become mobile and network-independent by not using their own pump. This allows the patient himself to become mobile. An anatomically adapted configuration of the evacuated vessel or the mentioned holder further advantageously makes it possible to inconspicuously wear the device on the patient's leg, for example. The evacuated vessel also makes no operating noise at all and is easy to use.

The hand-operated partial vacuum source also makes no operating noise and is easy to use. In the simplest case, the hand-operated partial vacuum source may be a plastic syringe with a sufficiently large volume. Other examples include a pump resembling a rubber ball, a bellows, and the like.

The wound covering element serves to close the wound so that a partial vacuum can be created. As an option, the wound covering element may be elastically configured (for example, by use of a silicone or an elastic polyurethane). In this way, when a partial vacuum is applied, the wound care device is pressed against the wound base producing a contact between the wound base and absorption body or wound contact layer. This alone is sufficient to stimulate an active acquisition of wound exudations from the depths of the wound. In this case, the main burden of acquiring the liquid is borne by the superabsorber. Furthermore, the full uptake capacity of the absorption body will likely be exhausted as the covering element yields to the liquid-dependent increase in volume of the absorption body.

In example implementations, the device for creating a subatmospheric pressure is accommodated in a flat housing. The flat housing is provided at least partly on its flat side facing the wound covering element with an adhesive surface, which in turn is covered with a peelable protective film element. After the protective film element is peeled off, a suction opening with check valve located at the flat side of the flat housing is released, coinciding with the opening on the wound covering element.

The flat housing is provided at least partly on its flat side facing the wound covering element with an adhesive surface, which in turn is covered with a peelable protective film element. After the protective film element is peeled off, the flat housing can be glued onto the wound covering element. The flat housing may preferably have a maximum thickness of 25 mm. In some implementations, the thickness may not be more than 22 mm, 20 mm, 18 mm, 15 mm, 12 mm, 10 mm or even 8 mm.

The device may include a liquid-permeable wound contact layer facing the wound. The absorption body taking up the wound exudations and the liquid-permeable wound contact layer facing the wound are preferably also permeable to air, so that the partial vacuum applied can also be extended into the wound region. Permeability to air may be provided using, for example, punchings, pores, slits or the like.

A permeable-to-air wound contact layer may be implemented as a plastic lattice (made for example from massive silicone or from a silicone-coated material), a foam plastic (that may be perforated, or in the form of an open-pore foam plastic known as Granufoam), a spacer fabric, a lattice having silver or copper wires or consisting of polymer fibers or wires containing silver or copper or their ions, a layer containing silicone or a hydrocolloid plus perforations, a layer containing cellulose, including derivates of cellulose such as carboxymethylcellulose or its derivates, and/or a perforated film.

A three-dimensional wound spacing grid may also be provided. An example of a three-dimensional spacing grid is available under the brand name Sorbion Plus™, and described in EP2004116A1. Such a wound spacing grid hinders granulation in the dressing and enables an atraumatic dressing change. A wound spacing grid also has a biofilm releasing action and a valve effect, so that reflux of exudate is reduced. The applicant has found that these properties, which are known from “passive” wound dressings, provide benefits in the “active,” or vacuum-supported wound dressings described herein.

The materials used for the mentioned wound contact layer may also be used on the side opposite the wound-contacting side directly beneath the wound covering element in order to distribute the partial vacuum evenly. A barrier may be formed between the device for creating subatmospheric pressure and the absorption body absorbing wound secretions. Such a barrier would not let any liquids pass assuring that no liquid gets into the pump. The liquid remains inside the wound covering and is taken up by the absorption body absorbing the wound exudations. The barrier is preferably formed with a semipermeable membrane, such as one made of a material like Goretex, etc. The barrier may also be a hydrophobic filter element, such as for example, one having a water column of 120 cm or more. A particle filter, which has for example a pore size less than 1 μm, may also be provided. The particle filter preferably has a pore size less than 0.2 μm. In this way, the evacuation of infectious particles can be prevented. The particle filter is preferably implemented in the liquid barrier.

The device for creating subatmospheric pressure may be arranged on the side of the wound covering element that is opposite the wound-contacting side. This configuration eases the replacement of the pump or the power supply. Control or power supply lines need not be led through the wound covering element minimizing contamination of the device for creating subatmospheric pressure and facilitating its reuse. In other implementations, the device for creating subatmospheric pressure is arranged on the side of the wound covering element facing the wound thereby presenting a more unified appearance and allowing the patient to perform tasks such as taking a shower, for example, while wearing the device on his body.

The device for creating subatmospheric pressure preferably has at least one pump. In example implementations, the device for creating subatmospheric pressure may include two or more pumps in parallel or in series. In a parallel arrangement, the delivered volume can be increased—even doubled. In a series arrangement, the maximum achievable partial vacuum can be increased—even doubled. For example, two pumps can each be able to create a maximum partial vacuum of −80 mm HG. When arranged in series, they can create a maximum partial pressure of −160 mm Hg. This is especially advantageous when using miniaturized pumps since these can often only generate a relatively small partial vacuum in solo arrangement, which might not be sufficient for clinical application. This arrangement advantageously does not result in a doubling of the power consumption of the device. There are other power consumers present in the device besides the pump, such as the control electronics or the interface. Therefore, a doubling of the power by a parallel or series arrangement of two pumps does not result in a doubling of the power consumption.

In another example implementation, the device for creating subatmospheric pressure comprises at least one control module. For example, a pressure sensor may be used to ensure that a sufficiently large partial vacuum is promptly restored by further pumping in the event of a loss of seal or a leakage. The control module may also be configured to be removable from the rest of the device and function as a remote control, for example.

In an example implementation, the device includes at least one battery. The type of battery used may be chosen from any of the following:

traditional disposable batteries

traditional rechargeable batteries

flat-pack batteries

arrays of small batteries, preferably “button cells”

flexible, rechargeable storage batteries

In an example implementation, traditional disposable batteries or traditional rechargeable batteries of types Lady, AAA or AAAA or CR ⅓ N. Other implemenations use arrays of small batteries or the flexible rechargeable storage batteries. Such batteries are based on polymers and paper, for example, and afford the greatest possible mechanical flexibility with only a few millimeters thickness. Such batteries may also be configured as extremely thin lithium ion storage batteries or metal hydride storage batteries and offer the advantage of having sufficiently high battery capacity with good flexibility and small thickness at the same time, which substantially increase the user comfort.

The device for creating subatmospheric pressure may be arranged directly on the wound covering element precluding the need for a separate vacuum hose, which presents problems in terms of manufacturing (sufficiently good rigidity so as not to collapse under vacuum) and also hygiene problems (danger of contamination). Such hoses are also often made to be too short.

In example implementations, the device includes a connection site for an outside power supply, which can be used, on the one hand, to recharge the rechargeable batteries or storage batteries of the device. The connection site may also be arranged for the device to be operated directly with power from this power supply. Providing power via the connection site advantageously powers the device using power from the outside power supply to provide the initial vacuum immediately after the device is put in place. Battery power may then be used to maintain the vacuum —which requires less power—during the operation of the device.

In an example implementation, the connection site may be a micro USB connection sleeve and an outside power supply may be a corresponding power pack. In this configuration, the connection sleeve may also serve as an interface for a PC in order to exchange data, such as for example, data regarding the operating state of the pump, an operating protocol of the pump, partial vacuum values, or even physiological values.

In another example implementation, the outside power supply may be a 220 V adapter or a 12V power supply.

In the broadest sense, the connection site for an outside power supply can also be a connection to a solar panel for operating the device for creating subatmospheric pressure and for recharging any rechargeable battery present.

In another example implementation, a coupling, a check valve and/or a three-way cock may be arranged between the device for creating subatmospheric pressure and the wound covering element or the absorption body taking up the wound exudations. These devices may be used to ensure that a) the device for creating subatmospheric pressure can be decoupled from the rest of the device, b) a partial vacuum once installed can be maintained for as long as possible, and/or c) a partial vacuum can be established initially or repeatedly via an outside pump or an outside vacuum vessel in order to spare the battery or when the battery is discharged.

The external pump or the external vacuum vessel may be available for example at a clinic or at the patient's house. The external pump or external vacuum vessel may also be configured as a mobile design (for example, in the form of a case, integrated in the garments of the patient or fastened to the patient's belt). The device should remain small and inconspicuous on the wound since its task is primarily to maintain the vacuum. When needed, the device provides sufficient pumping capacity to ensure an efficient therapy.

Furthermore, the device includes a sensor for at least one physiological and/or pathological parameter of the wound exudation. Preferred physiological and/or pathological parameters include:
a) pH value of the exudation
b) protein content
c) microbial load
d) percentage of blood or blood cells in the drained fluid, and/or
e) oxygen content.

Sensors that may be used include, for example, a pH meter, an oxygen probe, a germ detector, a hemoglobin sensor or a protein sensor. The sensors provide information that a doctor can use to take immediate necessary steps. For example, based on the sensor information, the doctor may administer antibiotics or topical application of antimicrobial agents (such as silver, copper, octenidine, antibiotics, etc.) or buffers, or administer suitable salt, Ringer or protein solution. If blood or blood cells are present in the drained fluid, an alarm can be sounded and/or the pumping process can be interrupted to prevent an acute anemia.

An example implementation of the device may also make recommendations for the wound flushing, and then perform the wound flushing, optionally after confirmation by the doctor. A flushing solution from a container can be introduced into the wound via the pump circulation. The flushing solution can contain, in particular, the above-mentioned agents or solutions.

An example implementation of the device may include a device for determining the microbial load and/or the material composition of the exudate. Such a device can be, for example, a biosensor (such as a biochip) or a test strip with color indicators. With respect to the microbial load, multiresistant germs are of special interest, particularly those of the type that are responsible for the notorious hospital infections such as MRSA (Methicillin-resistant Staphylococcus aureus, ORSA (Oxacillin-resistant Staphylococcus aureus), VISA (Vancomycin-intermediate Staphylococcus aureus) or VRSA (Vancomycin-resistant Staphylococcus aureus). Biosensors for such germs are based for example on the principle of the immunoassay or that of a biochip, preferably in the form of a disposable product. Biosensors may also be based on aptamers. Such technologies are known to the skilled person from the relevant literature.

With respect to the material composition of the exudation, serum proteins (albumens), matrix metalloproteases, clotting factors and proteins (such as thrombin, fibrin), inflammation markers (cytokines), insulin, or glucose are of special interest. Also of special interest are the typical markers for the kidney and liver values. Immunoassays may also be included, preferably in the form of a disposable product (test strip).

A biochip, preferably in combination with the operating and/or control element described below, may include a memory for storing and reading out the measured values for the microbial load and/or material composition of the exudation. In this way, the time variation of the composition of exudation can be tracked and recorded.

In an example implementation, the absorption body may be surrounded by a liquid-permeable sheath. The device may also be provided with a spacer body, which can be placed preferably between the wound and wound covering element. The spacer body may also be placed between the absorption body and wound covering element or between wound and absorption body if the device includes an absorption body.

The wound covering element in an example implementation is configured to be impermeable to liquid and/or gas making it possible to apply a vacuum, and to prevent the liquid, which might be contaminated, from emerging. The wound covering element is preferably elastic.

The wound covering element may include at least one adhesive device to enable application of the device to the patient's skin surrounding the wound. The adhesive device may also provide a liquid and/or gas-impermeable connection of the wound covering element to the skin. The adhesive device also ensures that a partial vacuum can be maintained. The adhesives that may be used include silicone glues, hydrocolloid glues, and the like.

The wound covering element should be permeable to water vapor. The wound covering element may also be transparent. The wound covering element may include a window for removal of the absorption body, which may be preferred in cases in which the wound is heavily exuding. Using the window, the absorption body can be changed and the device can remain on the patient.

The wound covering element may also include a means for securing the device for creating subatmospheric pressure. The means for securing may be, for example, an adhesive surface corresponding to the surface size of the device for creating subatmospheric pressure or its housing. Alternatively, a snap button or hook and eye fastener system can be provided.

The absorption body may include at least one superabsorbing substance, a modified cellulose and/or an alginate. Superabsorbing polymers (SAP) are plastics which are able to absorb a multiple of their own weight (up to 1000 times) in liquid. Chemically, superabsorbing polymers involve a copolymer of acrylic acid (propenic acid, C₃H₄O₂) and sodium acrylate (sodium salt of acrylic acid, NaC₃H₃O₂), wherein the ratio of the two monomers to each other can vary. In addition, a so-called core-cross linker (CXL) is added to the monomer solution, which joins (“cross links”) the long-chain polymer molecules formed to each other in places by chemical bridges. Thanks to these bridges, the polymer becomes water-insoluble. When water or aqueous salt solutions get into the polymer particle it swells up and stiffens this network on the molecular level, so that the water can no longer escape unaided. The superabsorbing polymers can be present in an example implementation of the wound care article of the invention in the form of a granulate, a powder, a loose filling, a molded piece, a foam, in the form of fibers, a fiber woven fabric, scrim, or nonwoven material and/or a fiber wadding.

Alternatively, superabsorbers based on methylacrylic acid, polyvinylalcohol/maleic anhydride copolymers, polysaccharide/maleic anhydride copolymers, maleic acid derivates, acrylamide/propane sulfonic acid copolymers, starch/acrylonitrile graft polymers, gelatinized starch derivates, alkyl or hydroxyalkylcellulose, carboxymethylcellulose, starch/acrylic acid graft polymers, vinyl acetate/acrylate copolymers, acrylonitrile or acrylamide copolymers can be chosen. In the present context, the germ inhibiting and matrix metalloprotease modulating properties of superabsorbing polymers as described in the literature are of special interest, since they synergistically support the action of the partial vacuum therapy.

In an example implementation, an absorption body from the Sorbion company, which contains the superabsorbent polyacrylatc, is equivalent in its germ-inhibiting action to an absorption body containing silver, which is used in a similar form in other partial vacuum therapy devices, but without the known side effects for silver.

Modified cellulose preferably involves derivates of cellulose, preferably sulfoalkylated cellulose and its derivates, preferably cellulose ethyl sulfonates (so-called “Durafaser”), carboxyalkylated cellulose, preferably carboxymethyl cellulose (so-called “Hydrofaser”), carboxyethylcellulose and/or carboxypropylcellulose, more complex cellulose derivatives such as sulfoethylcarboxymethylcellulose, carboxymethylhydroxyethylcellulose, hydroxy-propyl-methylcellulose, and amidated cellulose derivates like carboxymethylcellulose amide or carboxypropylcellulose amide. Carboxymethylcellulose is present especially in the form of sodium carboxymethylcellulose and is on the market by the name “Hydofaser”. In hygiene and wound care products, the fibers are converted into a two-dimensional matrix. By taking up liquid from the wound exudation, the fibers are gradually transformed into a gel cushion, which retains the liquid and does not release it again. The fibers here are constructed so that the wound exudation is taken up only in the vertical direction. This means that as long as there is enough capacity, the exudation does not flow beyond the wound margin. In this way, a maceration of the wound margin can be effectively prevented.

The mentioned hydroactive polymers may also include alginates. Alginates are obtained from brown algae and are woven into a fibrous fleece. Chemically, they are polysaccharides, specifically calcium and/or sodium salts of alginic acids. Alginates can absorb up to 20 times their weight in liquid, the wound exudation being stored in the cavities. The Ca2+ ions contained in the alginate lattice are exchanged for the Na+ ions from the exudation until the degree of saturation with Na ions is reached in the alginate. This results in a swelling of the wound dressing and a transformation of the alginate fibers into a gel body by swelling of the fibers.

The hydroactive polymers may also include hydrogel nanoparticles having hydroxy-terminated methacrylate monomers, such as 2-hydroxyethylmethacrylate (HEMA) and/or 2-hydroxypropylmethacrylate (HPMA), which are marketed as Altrazeal, for example.

In another example implementation, the absorption body has a fraction of≥40 wt. % of superabsorbing polymers. Especially preferably the weight fraction of the superabsorbing polymers is ≥45, 50, 55, 60, 65 or 70 wt. %.

The absorption body may also include a fleece containing cellulose fibers. The absorption body may be an essentially flat absorption body of absorption material consisting of an absorbent fleece with superabsorbing polymers distributed therein. These superabsorbing polymers may be present in the form of a granulate, a powder, a loose filling, a molded piece, a foam, in the form of fibers, a fiber woven fabric, scrim, or nonwoven material and/or a fiber wadding.

The absorption body includes at least one material chosen from the group containing a mat, especially an airlaid made of yarns or fibers of superabsorbing polymers with superabsorbing polymers worked into them, and/or a loose filling of superabsorbing polymers. The airlaid mat may include an essentially flat material segment of absorption material, which consists for example of an absorbent fleece of the mentioned fibers with superabsorbing polymers distributed therein.

This absorption body may correspond to the absorbent inlay contained in a wound dressing of the applicant, such as is disclosed in WO03094813, WO2007051599 and WO0152780, which are incorporated herein by reference, and marketed under the commercial brand name Sorbion Sachet™.

In another example implementation, the absorption body may also form a core having (possibly flakelike) fibers or yarns of superabsorbing polymers and superabsorbing polymers in granulate form. The granulates may be glued or welded to the fibers or yarns at several heights, and the granulates distributed over more than 50% of the overall construction height of at least one segment of the core, wherein mingled areas of granulate and fibers are present. The weight fraction of the superabsorbing polymers can lie preferably in the range between 10 and 25 wt. %. Similar constructions are known from traditional incontinence materials and as hygienic napkins for their cushioning properties. A sheath can be arranged around the core, where the sheath is overlapping in areas and covers a glue seam or portion thereof, for example.

The absorption body may also include a fleece, preferably a nonwoven or airlaid, which consists of superabsorbing fibers (“SAF”, preferably polyacrylate) or contains such a component. The fibers can be mingled with fluff pulp (cellulose) or with polyester fibers, for example. Alternatively or additionally, a layered construction can be provided.

The weight per unit area can lie in the range between ≥50 and ≤2000 g/m². Preferred are weights per unit area of 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, and/or 2000, each time +/−25 g/m².

The thickness can lie in the range between ≥2 and ≤50 mm. Preferred are thicknesses of 2, 4, 6, 8, 10, 12, 14, 16, 18, 20, 22, 24, 26, 28, 30, 32, 34, 36, 38, 40, 42, 44, 46, 48, and/or 50 each time +/−1 mm.

The uptake capacity can lie in the range between ≥3 and ≤30 ml of 0.9% salt solution/m² at 0.2 psi pressure. Preferred here are values of 3, 4, 5, 6, 7, 8, 9, 10, 11, 12, 13, 14, 15, 16, 17, 18, 19, 20, 21, 22, 23, 24, 25, 26, 27, 28, 29, and/or 30 ml of 0.9% salt solution/m². Alternatively the uptake capacity can lie in the range between ≥2 and ≤50 g of water/g. Preferred here are values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 g of water/g

The overall content of superabsorbing polymers can lie in the range between ≥5 and ≤100% w/w. Preferred here are values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100% w/w.

The tensile strength can lie in the range between ≥5 and ≤80 N/5 cm. Preferred here are values of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80 N/5 cm.

The extensibility can lie in the range between ≥10 and ≤80%. Preferred here are values of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80%.

In practice, the following types have proven to be especially advantageous:

| Type | 1 | 2 | 3 | 4 | 5 | 6 |
|---|---|---|---|---|---|---|
| Structure 1 | Layered | 40% | Bicomponent | Layered | 25% | 40% |
| | structure: | polyester | fiber of | structure: | polyester, | polyester |
| | thermobonded | short | SAF and a | thermobonded | 75% SAF | short |
| | airlaid with | staple | thermoplastic | airlaid with | | staple |
| | laminated | fiber; | | laminated | | fiber; |
| | nonwoven | 60% SAF | | nonwoven | | 60% SAF |
| Structure 2 | Bicomponent | Needle | Carded, | Bicomponent | Needle | Needle |
| | fiber of | felt | thermobonded | fiber of | felt | felt |
| | SAF and a | | nonwoven | SAF and a | | |
| | thermoplastic + | | | thermoplastic + | | |
| | Fluff-Pilp | | | Fluff-Pilp | | |
| Type of SAF | 101/6/10 | 102/52/10 | 102/52/10 | 101/6/10 | | |
| fiber | | | | | | |
| Weight (g/m²) | 560 | 540 | 1000 | 350 | 150 | 380 |
| Thickness | 6 | 5.4 | 20 | 3.5 | 2.4 | 3.8 |
| (mm) | | | | | | |
| Uptake | 31.21 | >20 g | >16 g | 19.51 | >25 g | >17 g |
| capacity | water/m² | water/g | water/g or | water/m² | 0.9% salt | water/g or |
| | | | 16000 g | | solution/g | 6400 g/m² |
| | | | water/m² | | | |
| Uptake | 16 | | | 16 | | |
| capacity under | | | | | | |
| pressure (ml of | | | | | | |
| 0.9% salt | | | | | | |
| solution/m² at | | | | | | |
| 0.2 psi | | | | | | |
| pressure) | | | | | | |
| Total content of | 18 | 40 | 50 | 18 | 75 | 60 |
| superabsorbing | | | | | | |
| polymers (% | | | | | | |
| w/w) | | | | | | |
| Tensile strength | | | | | 16 ± 13 | 16 ± 13 |
| (N/5 cm) | | | | | | |
| Extensibility | | | | | 60 ± 18 | 60 ± 18 |
| (%) | | | | | | |

The absorption body in another embodiment can likewise contain at least one flat layer having fibers or yarns of superabsorbing polymers, to which superabsorbing polymers in granulate form are glued. This yields a structure of the body in an example embodiment that has at least three layers, wherein two cover layers enclose a layer having superabsorbing polymers.

There are no interminglings of fibers and superabsorbing polymers in the plane, but only fixed proximities of the two materials. In one example embodiment, several layers when present are physically compacted together by rolling, pressing, calendering or similar methods.

Parameter ranges similar to the above are preferred. The liquid retention can be between ≥5 and ≤100 g/g. Preferred are values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 g/g

In practice, the following types have proven to be especially advantageous:

| Type | 1 | 2 | 3 | 4 | 5 | 6 | 7 | 8 |
|---|---|---|---|---|---|---|---|---|
| Weight | 450 | 300 | 150 | 50 | 100 | 120 | 140 | 440 |
| (g/m²) | | | | | | | | |
| Thickness | 1.3 | 1.2 | 0.9 | 0.7 | 0.7 | 0.76 | 1 | 1.2 |
| (mm) | | | | | | | | |
| Liquid | 28 | 33 | 28 | 15 | 25 | 28 | 11.5 | 38 |
| retention | | | | | | | | |
| (g/g) | | | | | | | | |
| Tensile | 25 | 55 | 20 | 20 | 20 | 20 | 15 | 20 |
| strength | | | | | | | | |
| (N/5 cm) | | | | | | | | |
| Uptake | 45 | 20 | 50 | 20 | 40 | 50 | 28 | 55 |
| capacity | | | | | | | | |
| (g/g) | | | | | | | | |

Furthermore, the body can have recurring patterns or graining, such as a checkered pattern, a punched pattern, or the like. Preferably, the absorption body has a mass per unit area of 5×10, 5×20, 10×10, 10×15 or 15×15 cm.

In an example implementation, the absorption body has, besides a layer having superabsorbing polymers, at least one second flanking layer which has less or no superabsorbing polymers and which protrudes beyond the area of the aforementioned layer. In this way, it is ensured that the layer having superabsorbing polymers can grow in volume according to the liquid uptake, without the volume increase being noticeable on the outside, because the latter is lined by the second layer.

An example implementation of the device for creating subatmospheric pressure is reversible in its pumping direction. In this way, the device can also be used as a controllable dispensing pump for medications, flushing solutions, etc.

The device for creating subatmospheric pressure may also include a display or operating state indicators, for example, in the form of a LCD, LED or TFT display or in the form of LED lights. Such displays can display, for example, operating state, battery state, or error messages, or depict other parameters.

It is preferably provided that the device for creating subatmospheric pressure or its control module can be operated from at least one of the following devices:
touch screen
membrane keyboard
Reed contact
Remote control.

The touch screen, the membrane keyboard, and also the Reed contact advantageously allow for better hygiene, since the first two are easier to keep hygienic than a traditional keyboard and the latter lacks any accessible contacts or lines. The remote control may operate, for example, using the IR standard, or the Bluetooth standard, and may correspond in its functioning to a radio remote control.

The touch screen can be implemented so that it is integrated seamlessly in the housing and only becomes visible to the user when activated. The touch screen or the display can also be flexible or bendable. The device for creating subatmospheric pressure or its control module may also include a foldable or slidable operating unit or a corresponding storage battery or battery similar to that of a mobile telephone. This has hygienic benefits, in addition to aesthetic ones. Two levels of operation can be implemented in this way, namely, one operating level intended for the patient, which makes the basic functions available, and one operating level intended for the upkeep personnel or service technicians, which provides further operating and maintenance functions.

The wound covering element may include a marking, which corresponds to the surface area of the device for creating subatmospheric pressure or its housing.

The device for creating subatmospheric pressure may be configured to generate excess pressures in a reversed operating mode, which may be useful in a temporary excess pressure therapy, such as for wound compression and/or to stop bleeding. The device may also include means for dispensing medications or introducing flushing medium.

In another aspect of the invention, a method is provided for the operation of a wound care device. An example method, a given partial vacuum is first generated with the help of the device for creating subatmospheric pressure. The device for creating subatmospheric pressure is then switched off, and only when a partial vacuum threshold is crossed above or below is the device for creating subatmospheric pressure again switched on and generates a given partial vacuum.

In another example implementation a method is provided for using a wound care device for treatment of wounds having soft tissue defects, infected wounds after surgical debridement (so-called “maintenance debridement”), active wound debridement, lymph fistulas, sternal wound infections, thoracic wall windows, decubitus, ulcus cruris, chronic wound healing disorders, radiation ulcer, abdominal compartment syndrome, septic abdomen, enteral fistulas and/or wounds that are caused by one or more edemas, and/or for the fixation of skin transplants and/or for wound conditioning. What is meant here in particular is the treatment of inflammatory edema or edema caused by chronic venous insufficiency.

The effects exerted by the partial vacuum therapy are based in part on so-called “macrostrain” and “microstrain” effects (macrodeformation and microdeformation). Under partial vacuum, the wound care device exerts mechanical and biological forces on the wound via its wound contact layer (such as a silicone lattice, a foam, a cellulose fleece or a three-dimensional wound spacing grid). This creates a milieu which promotes the wound healing. Macrodeformation is the visible change which occurs when the partial vacuum contracts the foam plastic. Macrodeformation draws the edges of the wound together, produces direct and comprehensive contact between wound bed and wound contact layer, distributes the partial vacuum uniformly and removes exudation and infectious material (debridement). Microdeformation takes place on the cell level. The cells are stretched. Microdeformation reduces the formation of edema, promotes blood flow, increases cell proliferation and migration and promotes the formation of granulation tissue.

In another example implementation a method is provided for using a wound care device for postoperative wound or suture care. This approach, also known as incision management, reduces the risk of postoperative complications. The therapy helps the wound edges hold together, which in turn lessens the likelihood of suture dehiscence. The therapy also reduces OP-related tissue stress and edema and protects the wound from external contamination. These benefits especially come into play during outpatient use, in the area of trauma medicine or in military field use.

In another example implementation, the wound care device may also be used in conjunction with a compression dressing. The term “compression dressing” refers herein to a textile, elastic bandage. Various compression techniques are distinguished (Putter, Fischer, spiral reverse wrapping). Compression of the tissue has the following effects:
constriction of suprafascial veins with restoration of valve function
increasing the tissue pressure with higher resorption of tissue fluid in the lymph vessels, and
improvement of the muscle pump (by strengthening the abutment for the muscles)
indications are, for example, edemas, primary varicosis, thrombophlebitis, postthrombotic syndrome, ulcus cruris

Applying a compression dressing requires short-stretch bandages, bandage clips, scissors and optionally padding material. After hygienic disinfection of the hands, the required materials are prepared and the patient is laid on his back. In the beginning, the limbs must be lifted up or optionally milked in the direction of the heart in order to prevent a venous blood congestion. Bone protrusions such as shinbone, ankle, and depressions such as the pit of the knee can be cushioned appropriately. The foot should now be placed in a 90° position at the ankle joint. As shown in FIG. 26, during the wrapping, the heel should also be wrapped, or else a window edema may occur. The compression should decrease uniformly from distal to proximal direction. During the wrapping, the bandage should be stretched up to ¾ of the maximum extensibility. Each bandage should overlap the next one around halfway. At joints, the wrapping can be done as a FIG. 8 around the joint, in order to prevent folds from forming (spiral reverse wrapping) as shown in FIG. 26. An example implementation of the wound care device can now be worked into the compression dressing with or without the device for creating subatmospheric pressure in place. In this way, a synergy can be achieved between compression and partial vacuum therapy. In the event that the optional remote control of the device for creating subatmospheric pressure option is being used, a constant monitoring of the partial vacuum therapy can be assured even when the device for creating subatmospheric pressure is not accessible.

Further embodiments will be illustrated below by the figures and experiments, although their specific configuration in no way limits the invention.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 is a schematic sectional representation of an example of a wound care device that uses subatmospheric pressure;

FIG. 2 is a schematic sectional representation of another example of a wound care device having a foam absorption body;

FIG. 3 is a perspective bottom view of a micropump;

FIG. 4 is a schematic top view of the upper side of the micropump of FIG. 3;

FIG. 5 is a schematic top view of the upper side of another embodiment of the micropump;

FIG. 6 is a diagram of a marking on the wound covering element;

FIG. 7 is a schematic sectional representation of another embodiment of the wound care device;

FIG. 8 is a diagram of a mini-USB connection on the micropump,

FIG. 9a is an exploded view of a flat adapter;

FIG. 9b shows the flat adapter pressed together across the wound covering element;

FIG. 9c depicts the mounting of the micropump on the flat adapter with a schematically indicated absorption body;

FIG. 10a is a schematic sectional representation of a magnetic connection of the micropump to the flat adapter;

FIG. 10b is a schematic sectional representation of another embodiment of the magnetic connection, with a micropump situated inside the absorption body;

FIG. 11a is a top view looking at the wound covering element of another embodiment of the wound care device, with divided fields of the wound covering element;

FIG. 11b depicts section A-A of FIG. 11a;

FIG. 12 is a top view looking at the wound covering element of another embodiment of the wound care device, likewise with divided surface of the wound covering element;

FIG. 13a is a top view looking at the wound covering element of another embodiment of the wound care device;

FIG. 13b is a schematic side view of the wound care device of FIG. 13a;

FIG. 14 is a top view of a wound covering element having flat batteries;

FIG. 15 is a schematic top view of another embodiment of the wound covering element with flat battery;

FIG. 16a is a top view looking at a round wound covering element of another embodiment of the wound care device;

FIG. 16b is a schematic sectional representation the wound care device of FIG. 16a;

FIG. 17a is a perspective view of a foamlike absorption body;

FIG. 17b is a sectional representation of the absorption body of FIG. 18a;

FIG. 17c depicts the absorption body of FIG. 18b with a micropump mounted in it;

FIG. 17d is a sectional representation of a wound care device having the foamlike absorption body and micropump;

FIG. 18 is a sectional representation of another embodiment of the wound care device;

FIG. 19a is a top view of another embodiment of the wound care device, having a drainage collector;

FIG. 19b depicts section B-B of FIG. 19a;

FIG. 19c is a top view of the wound care device of FIG. 19a looking at its side facing the wound;

FIG. 20 is a schematic sectional representation of another embodiment of the wound care device having a three-way valve;

FIG. 21 is a top view of another embodiment of the wound care device having two micropumps looking at the wound covering element.

FIG. 22 is a schematic sectional representation of another embodiment of the wound care device having a suction head arranged between the micropump and the wound covering element;

FIG. 23 is a schematic sectional representation of the foamlike absorption body shown in FIG. 17a, with several suction heads;

FIG. 24 depicts a wound care device with the built-in absorption body of FIG. 23;

FIGS. 25a and 25b are flowcharts illustrating operation of a sample operating mode of a device according to the invention, and;

FIG. 26 depicts a compression bandage in which the device according to the invention can be used.

### DETAILED DESCRIPTION OF THE INVENTION

The foregoing and other features and advantages of the invention will become more apparent from the following detailed description of exemplary embodiments, read in conjunction with the accompanying drawings. The detailed description and drawings are merely illustrative of the invention rather than limiting, the scope of the invention being defined by the appended claims and equivalents thereof.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

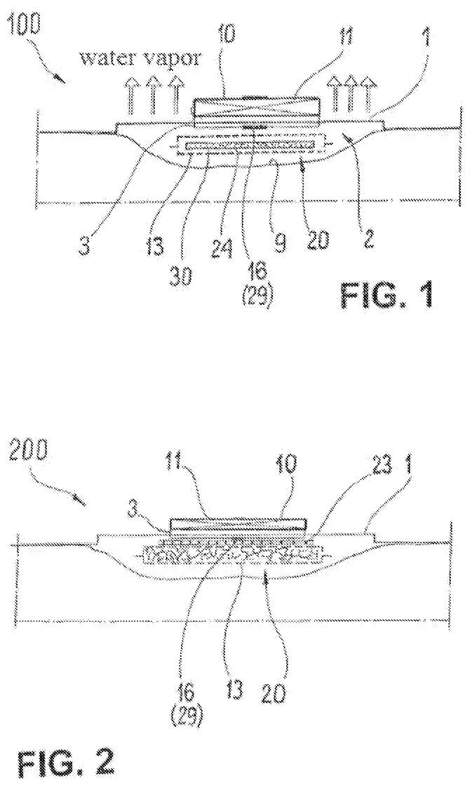

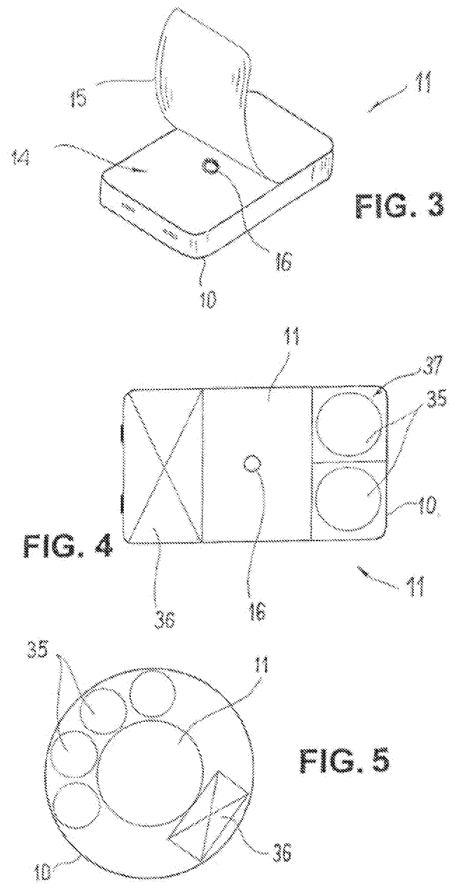

FIG. 1 is a schematic sectional representation of an example of a wound care device 100 that uses subatmospheric pressure. The wound care device 100 includes a liquid-impervious and water vapor-pervious wound covering element 1, an enclosed absorption body 20 and a device for creating subatmospheric pressure. The device for creating subatmospheric pressure shown in FIG. 1 is a micropump 11. The wound covering element 1 is configured as a flexible film-like membrane. The micropump 11 includes a rectangular flat housing 10, which is provided with a suction opening 16 (see FIG. 3); the suction opening 16 shown in FIG. 1 comprising a check valve 29.

The micropump 11 in FIG. 1 may be implemented using a piezoelectric membrane pump, which has a low power consumption, compact construction, and very small dimensions, enabling the use of the micropump in portable systems. The micropump 11 may advantageously be arranged directly on the wound covering element 1, since it has a very low weight. The micropump 11 in FIG. 1 plus control unit and optional battery (batteries) may be accommodated in a flat housing 10.

As shown in FIG. 1, the micropump 11 (which may be of a type manufactured by Bartels Mikrotechnik GmbH, Dortmund, Germany) is arranged on the side of the wound covering element 1 away from the wound and detachably glued to the wound covering element 1. This allows the micropump 11 to be reused if needed, as long as safety precautions such as sterilization are taken. FIG. 1 also depicts a wound cavity 2 bounded by the wound covering element 1 glued onto the patient's skin, and a wound base 9. The absorption body 20 located in the wound cavity 2 includes a segment 30 of a fleecelike textile mat fortified with a superabsorbing substance 24, and a sheath 13.

FIG. 2 shows a similar wound care device (reference number 200) in which the sheath 13 includes a foamlike inner core instead of the textile mat. A spacer body 23 disposed between the wound covering element 1 and the absorption body 20 allows gases and air to pass through.

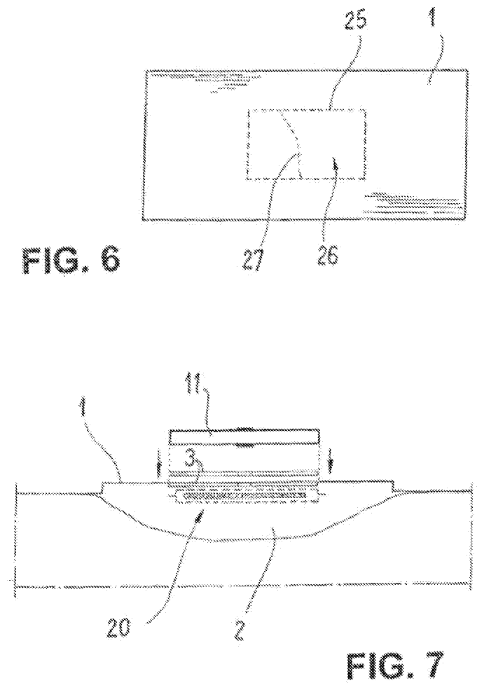

Examples of the placement of the micropump 11 are shown in FIGS. 6 and 7. The wound covering element 1 has an adhesive surface 26 corresponding to the surface area of the flat housing 10. The adhesive surface 26 is covered by a peelable protective film element 27. A marking 25 surrounds the adhesive surface 26 to mark where on the wound covering element 1 (sec FIG. 7) the flat housing 10 of the micropump 11 is to be applied.

The micropump 11 is shown in FIGS. 3, 4 and 5 in a schematic magnified view. The flat housing 10 may be removably placed on the wound covering element 1 by first peeling off a protective film element 15 revealing an adhesive surface 14 and the suction opening 16. As shown in FIG. 4, the rectangular flat housing 10 is divided into three inner compartments, which include a control module 36, the micropump 11 itself, and a battery compartment 37. The battery compartment 37 holds two button cells 35. FIG. 5 depicts an example of a round flat housing, on which four button cells 35 and the control module 36 are arranged at the periphery.

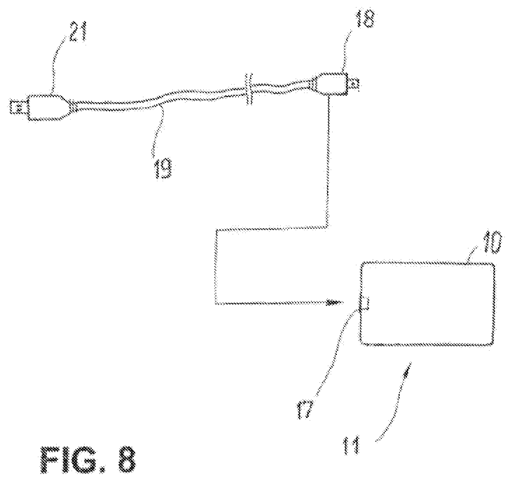

As shown in FIG. 8, the flat housing 10 of the micropump 11 may be outfitted with a mini-USB connector sleeve 17 in which to plug a mini-USB connector 18. The mini-USB connector sleeve 17 is in turn connected via a cable 19 to a USB connector 21 for a PC, or a connector for a cigarette lighter. The connection scheme may resemble that of a typical navigation device. It would be advantageous for the connector to the PC to allow an immediate evaluation, such as analysis of the wound exudation, when the micropump is outfitted with a measurement sensor (not shown).

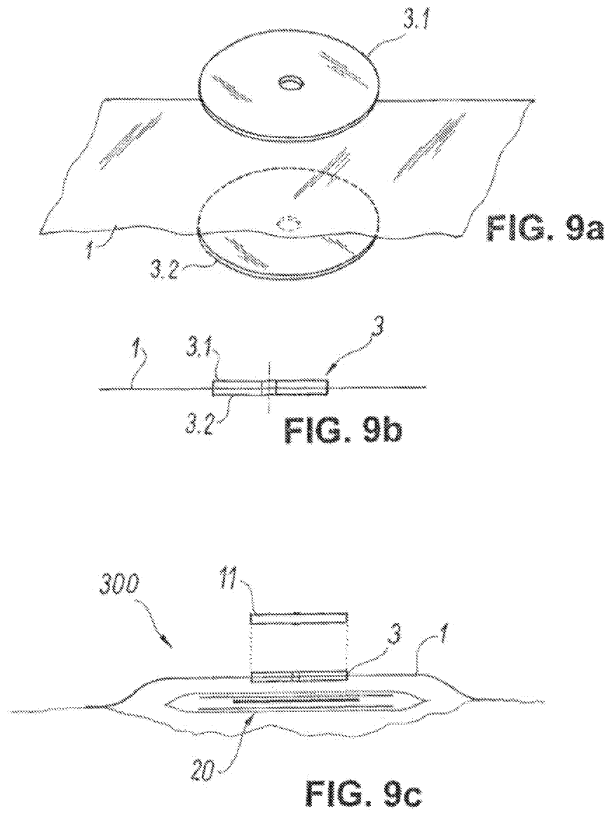

Another wound care device 300 is shown in FIG. 9c. The micropump 11 lacks a hose, but is in contact with the wound cavity 2 via a flat adapter 3, so that it is not directly connected to the wound covering element 1 (see FIGS. 9a and 9b). The flat adapter 3 consists of two congruent disks 3.1, 3.2, which can be placed on the wound covering element 1 from “above” and “below.” At least one of the two disks 3.1, 3.2 of the flat adapter 3 may be made of plastic, metal, or magnetic foil.

The disks 3.1, 3.2 can be locked or glued together across the wound covering element 1. However, in terms of tightness, a glue connection is preferred.

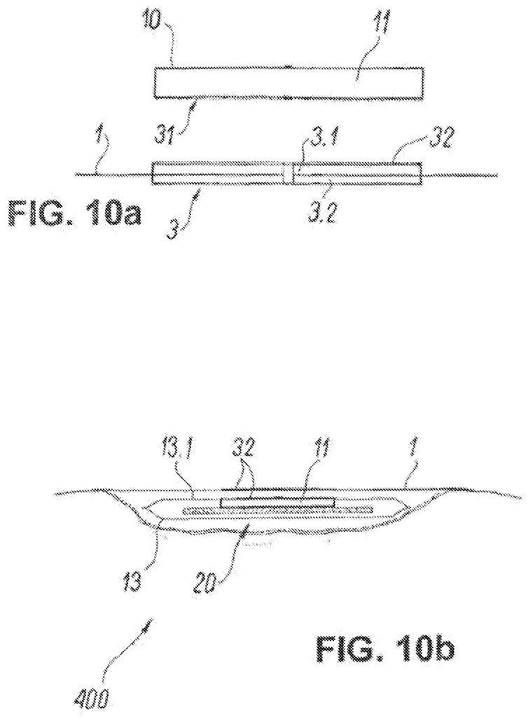

If the upper disk 3.1 is magnetic, the flat housing 10 of the micropump, which is coated with a very thin magnetic foil or with magnetic powder, can simply be laid on the flat adapter 3. In the present case (see FIG. 10a), the flat housing 10 is provided on its side facing the wound covering element 1 with a magnetic powder layer 31 and the upper disk 3.1 with a magnetic foil 32. The flat adapter 3 advantageously improves the positional stability of the removable micropump 11 and simplifies its mounting on the wound covering element 1.

FIG. 10b depicts another configuration of the magnetic connection. In the wound care device 400 shown in FIG. 10b, the micropump 11 lies beneath an upper sheath segment 13.1 of the sheath 13 of the absorption body 20 facing the wound covering element 1. This allows the micropump 11 to be arranged beneath the skin level at the wound at least in the initial phase of the wound therapy. A pliable wound spacing grid 33 (product SORBION PLUS, manufacturer: SORBION AG, Senden, Germany) is also arranged beneath the absorption body 20. A meshlike textile fortified with silver or copper may optionally be used instead of the wound spacing grid 33. The upper flat side of the flat housing of the micropump 11 and the wound covering element 1 are each provided with magnetic foil 32. A locking connection (not shown) may optionally be used in place of the magnetic foil.

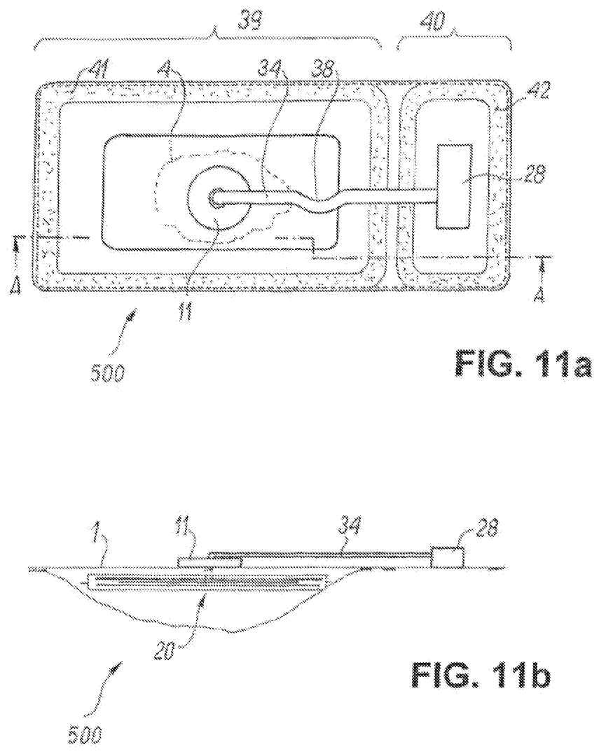

FIG. 11a and 11b depict a wound care device 500 comprising the wound covering element 1, the micropump 11, and a storage battery 28. The wound care device 500 includes a cable 34 situated in between and electrically connecting these components. The cable 34 has an arc-shaped compensation segment 38 to compensate for changes in length when the volume of the absorption body 20 increases. The wound covering element 1 is divided into a wound treatment area 39 and a battery area 40 separated from each other by peripherally encircling adhesive surfaces 41, 42. This permits both the micropump 11 and the storage battery 28 to be mounted in advance on a wound covering element 1. The storage battery 28 lies outside the wound treatment area 39, and in particular, outside of a wound contour 4.

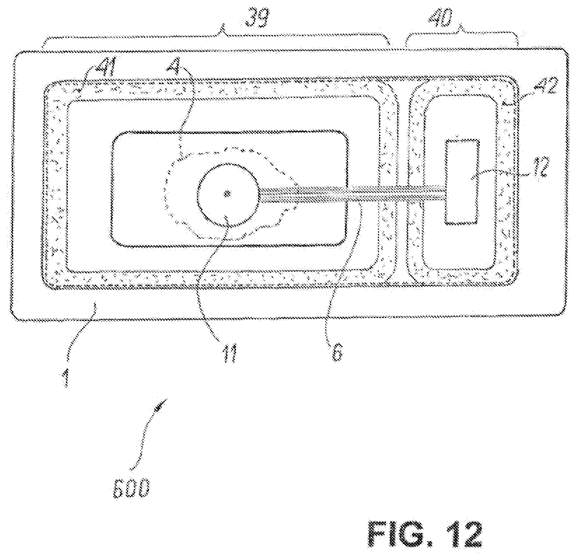

FIG. 12 illustrates a wound care device 600 similar to the wound care device 500 shown in FIGS. 11a and 11b. The same components are marked with the same reference numbers. With respect to the wound dressing 600 in FIG. 12, an electrical connection of the micropump 11 to a lithium ion battery 12 is provided by a foil-like printed circuit 6.

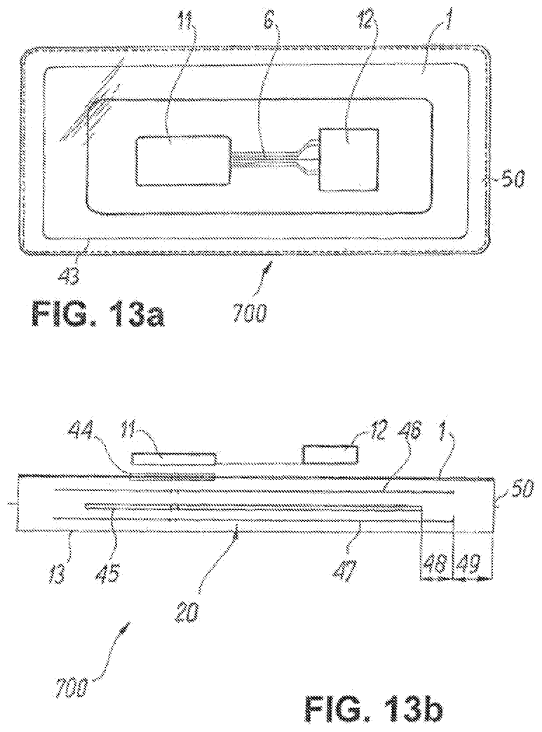

FIGS. 13a and 13b show a wound care device 700 similar to the wound care device 600 shown in FIG. 12. The wound care device 700 in FIGS. 13a and 13b include the micropump 11, the lithium ion battery 12, and printed circuit 6 arranged inside a contour 43 of the absorption body 20. The micropump 11 is locked by a rectangular flat adapter 44, built into the wound covering element 1.

As shown in FIG. 13b, the absorption body 20 has the mentioned sheath 13. Two superabsorbing textile segments 46, 47 are arranged inside the sheath 13 and in between a smaller sheetlike superabsorbing cellulose mat 45. The differences in area between the textile mats and an encircling seam 50 of the sheath produce desired expansion spaces 48, 49.

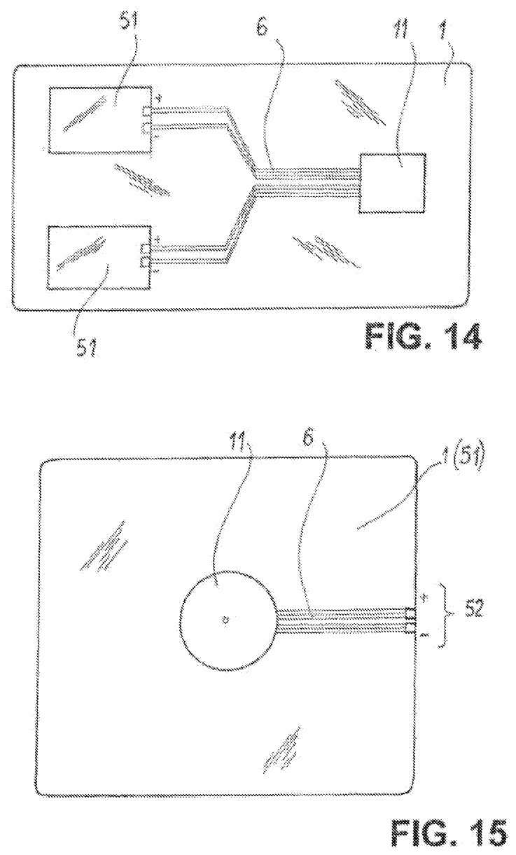

Referring to FIG. 14, another wound covering element 1 has two elastic foil-like flat batteries 51, which are glued onto the wound covering element 1 and which arc electrically connected via the printed circuit 6 to the micropump 11. The flat battery constitutes a new development of electrically conducting polymer foil, which is known in the technical journalism as a “paper battery.”

The wound covering element 1 shown in FIG. 15 with a format of 10 cm×10 cm consists of an electrically conducting, thin, bendable polymer foil. For this reason, the wound covering element 1 functions like a flat battery 51. By the printed circuit 6, the micropump 11 centrally arranged on the flat battery 51 is connected to poles 52 (plus, minus), which are embedded in the polymer foil.

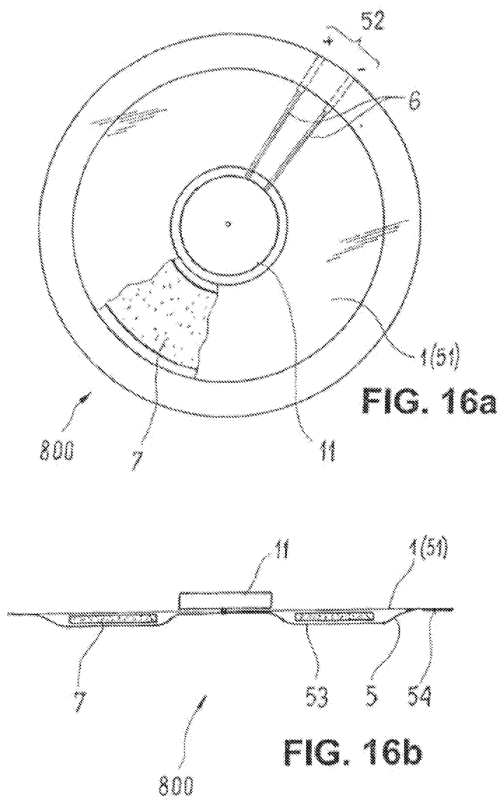

FIGS. 16a and 16b show a flat, circular wound care device 800 having an annular sheathed absorption body 7, the centrally arranged micropump 11 and the printed circuit 6. The wound covering element 1 is implemented using a circular flat battery 51. The micropump 11 is electrically connected to the poles 52 across the printed circuit 6. The wound covering element 1 and an annular film segment 53 facing the wound forms a sheath 5, which has a peripheral adhesive surface 54.

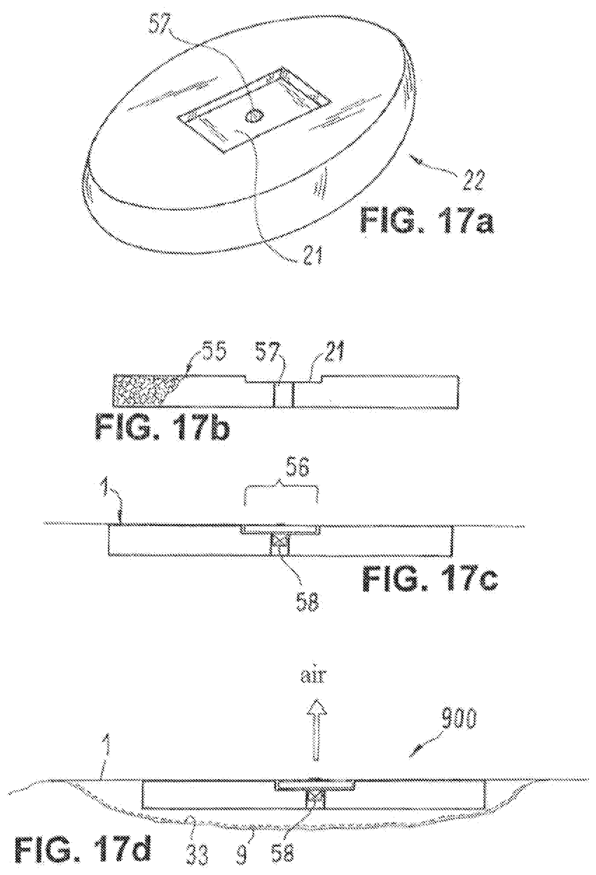

Another wound care device 900 is shown in FIG. 17d. The wound care device 900 has a foamlike absorption body 22, made of polyurethane, in the middle of which is cut out a rectangular seat 21 (see FIGS. 17b and 17c) to accommodate the micropump 11, such that the upper flat side of the micropump 11 is flush with a surface 55 of the foamlike absorption body. Moreover, a release-adhesive region 56 is provided on the wound covering element 1, which roughly coincides with the seat 21. The other region of the wound covering element 1 is not joined to the absorption body 22. In a through opening 57 of the absorption body 22 is placed a check valve 58. Between the absorption body 22 and the wound base 9 lies the wound spacing grid 33.

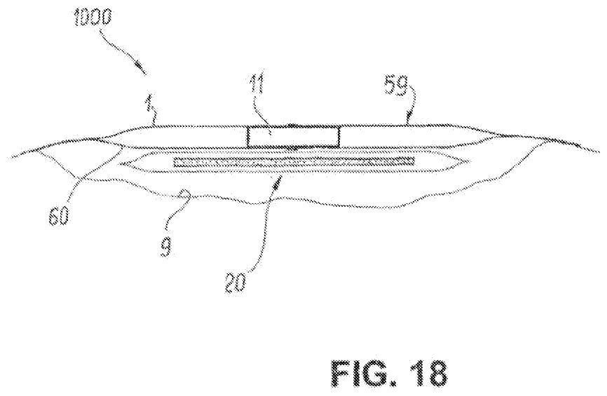

FIG. 18 shows a wound care device 1000 in which the micropump 11 is placed inside a foil-like sheath 59 formed by the wound covering element 1 and a “lower” sheath segment 60 facing the wound. The lower sheath segment 60 is permeable to gas and liquid, while the wound covering element 1 is only permeable to water vapor. The absorption body 20 lies beneath the sheath 59.

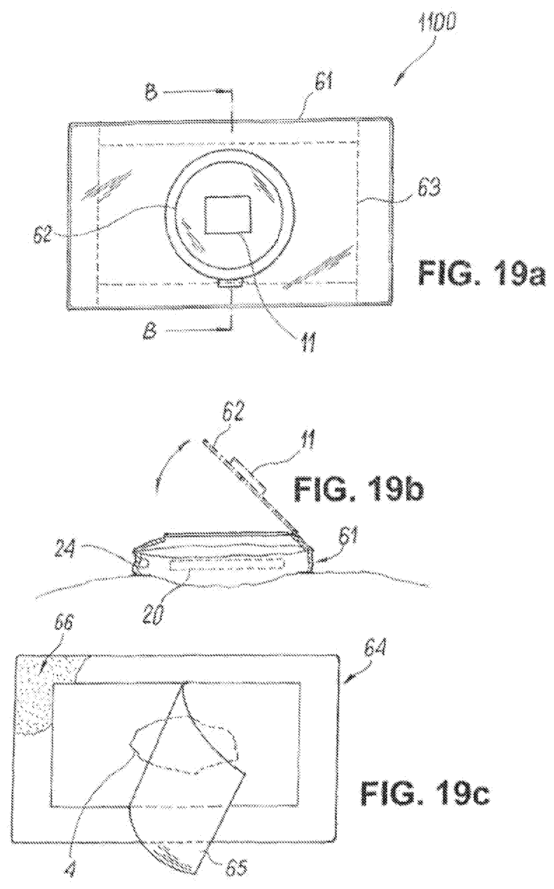

FIGS. 19a to 19c show a pouchlike wound care device 1100 having a pouch 61 in the manner of a familiar drainage collector with a swiveling window flap 62 (see FIG. 19b). The pouch 61 is folded together at its periphery 63. The micropump 11 is arranged in the middle on the window flap 62 and electrically connected to a battery, not shown. The battery function can be provided by the transparent foil element (flat battery) of the window flap 62 (not shown). The inner surface of the pouch 61 is lined or coated with the superabsorbing substance 24 (see FIG. 19b). The superabsorbing substance 24 can be gel-like or interspersed in a textile material.

As shown in FIG. 19c, the pouch 61 has a foil-like, glue-coated bottom 64, which is cut out according to the wound contour 9, so that a central bottom segment 65 can be peeled off and the bottom 64 glued by its peripheral adhesive surface 66 to the skin of the patient. Inside the pouch 61 lies the absorption body 20, which can be removed through the swiveling window flap 62 installed therein. Another embodiment (not shown) has a bottom made from the mentioned wound spacing grid 33 (product SORBION PLUS, manufacturer: SORBION AG) and provided with an encircling adhesive surface.

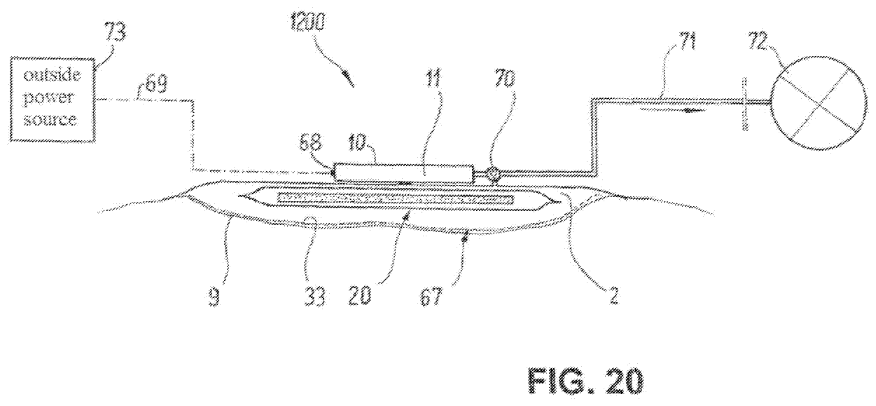

FIG. 20 shows another example of a wound care device 1200, which is similar to the wound care device 100 shown in FIG. 1. The flat housing 10 of the micropump 11 is arranged on the side of the wound covering element 1 away from the wound and removably glued to wound covering element there. Beneath the sheathed absorption body 20 lies the wound spacing grid 33, which is oriented with its smooth surface 67 toward the wound base 9. At first the wound spacing grid 33 and then the absorption body 20 (products: SORBION PLUS and SORBION SACHET, manufacturer: SORBION, Senden, Germany) are placed on the wound base 9. The foil-like wound covering element 1 is glued tightly to the skin of the patient around the wound.

The micropump 11 is removably glued to the wound covering element 1. The micropump 11 contains the batteries of the type described with reference to FIG. 4. The micropump 11 is additionally connected across a coaxial connector 68 and cable 69 to an outside power source 73. The energy source can be a storage battery or power mains.

The micropump 11 is connected across a three-way valve 70 and vacuum line 71 to an outside vacuum pump 72. The micropump 11 is first blocked by the three-way valve 70. The micropump 11 is not working. By activating the outside vacuum pump 72, the air is almost completely evacuated from the wound cavity 2. By adjusting the three-way valve 70, the outside vacuum pump 72 is automatically shut off. At this time, the micropump 11 takes over the suction function. The partial vacuum achieved by the outside vacuum pump 72 is maintained by the micropump 11. The system can be outfitted with a programmed interval switching, which sets the micropump 11 in motion as needed. This interval switching can be connected to the control module or be part of the control module.

The control module can be remote controlled (for example, by the patient or nursing personnel). The remote control can be activated by a traditional remote control.

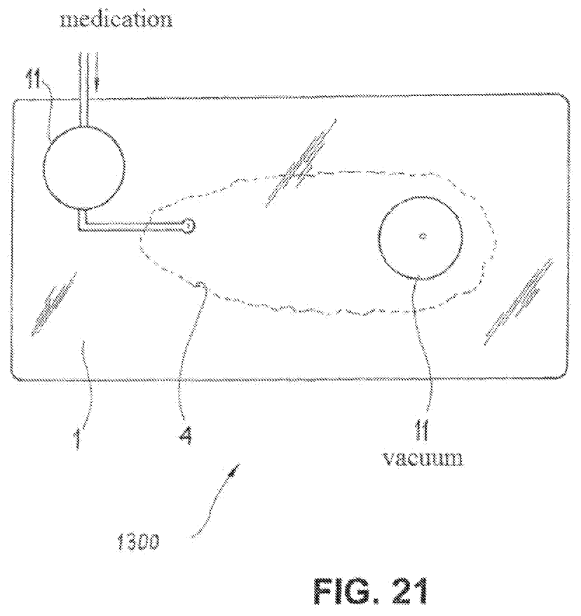

FIG. 21 shows another example embodiment of the wound care device 1300. In the wound care device 1300 in FIG. 21, the absorption body is left out for clarity of the drawing. Two micropumps 11 are arranged on the wound covering element 1. One of the micropumps 11 operates as the vacuum pump and the other operates as a dispensing pump for medication or provides a flushing function. The micropumps 11 are reversible. The valves on the micropump determine in which direction the fluids are delivered from the pump.

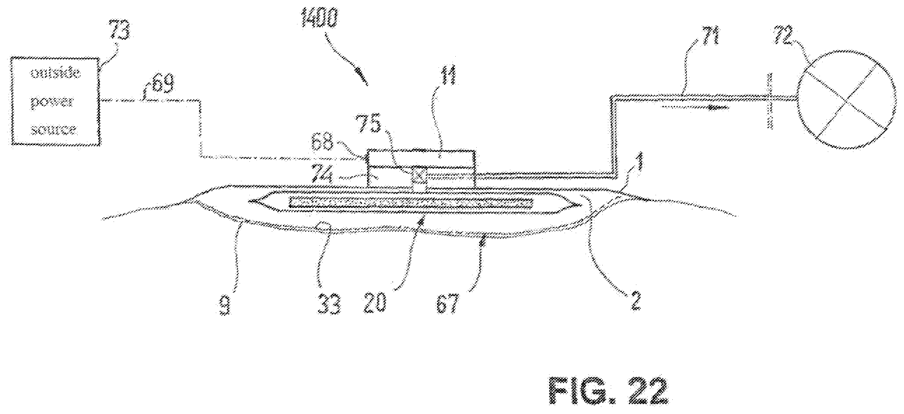

FIG. 22 shows a wound care device 1400 in which a suction head 74 is fastened to the wound covering element 1. The micropump 11 sits on the suction head 74 and not on the wound covering. The micropump 11 can be removably fastened by a locking or snap connection, by a glue connection, or magnetically to the suction head 74. The suction head 74 is provided with a three-way valve 75, to which the outside vacuum pump 72 is connected via the 71 vacuum line. The schematically represented suction head 74 can be highly flattened. Otherwise, the wound care device 1400 is characterized by the same construction and the same function as already described in FIG. 20.

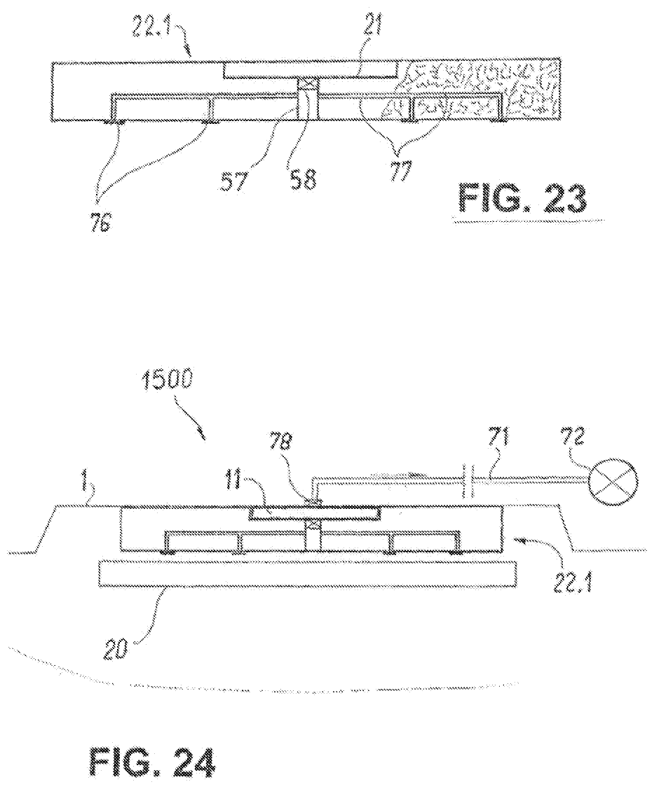

FIG. 23 shows an absorption body 22.1 made from a soft polyurethane foam, similar to the absorption body 22 (see FIG. 17). The difference between the two absorption bodies 22 and 22.1 is that the latter is outfitted with several small suction heads 76 distributed on its underside, each of which is connected by a line 77 to the opening 57 worked into the foam with a check valve 58. The “mini suction heads” contribute to an improved pressure distribution.

The absorption body 22.1 according to FIG. 24 is part of a wound care device 1500. The wound care device 1500 furthermore contains the mentioned wound covering element 1, the micropump 11 placed in the seat 21 of the absorption body 22.1, and the absorption body 20 facing the wound base (not shown). The foamlike absorption body 22.1 has an essentially smaller suction force than that of the absorption body 20. Furthermore, it plays the role of a buffer between the micropump and the lower absorption body 20. A small-pore membrane, not shown, can also lie between the two absorption bodies 22 and 22.1. The micropump 11 is connected to the outside vacuum pump 72 across a three-way valve 78 arranged on the micropump and the vacuum line 71. The micropump 11 can have at least one button cell and/or be connected to an outside power source (see FIG. 20).

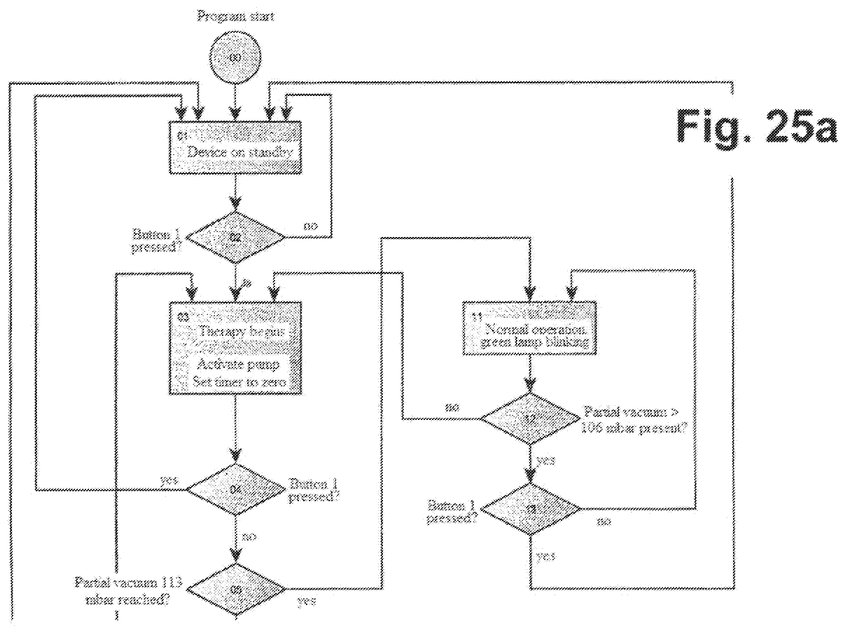

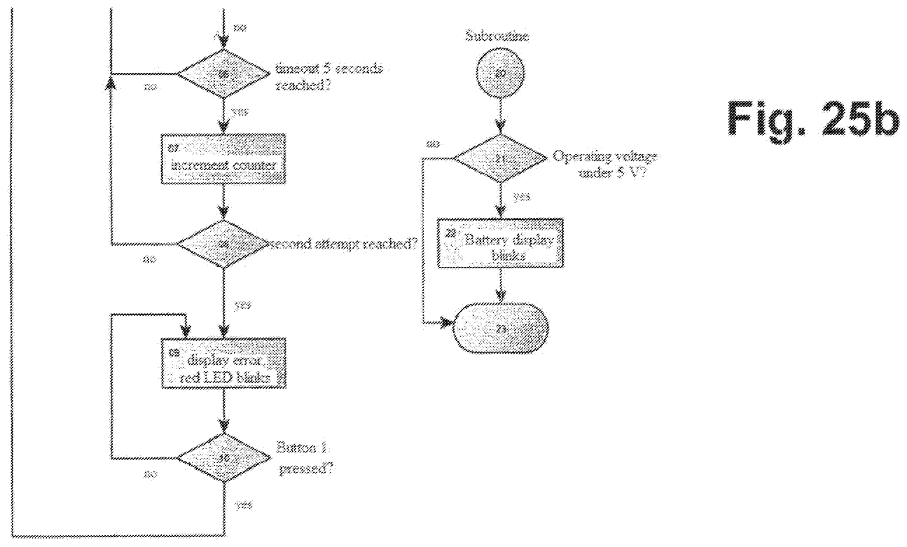

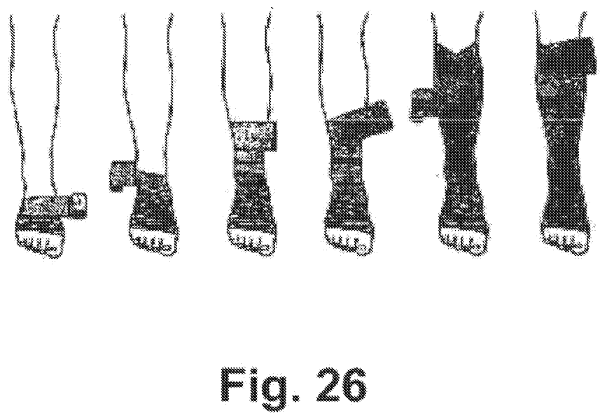

FIGS. 25a and 25b illustrate operation of an example method for an example operating mode of a device according to the invention. The method illustrated in FIGS. 25a and 25b may be performed using an example of the wound care device that includes a control module and a user interface having buttons to allow the user to enter control function. FIGS. 25a and 25b illustrate operation of the device in attaining a pressure of around 106 mbar to 113 mbar. It is noted that the pressure levels indicated are shown as examples and that operation of the method may involve any other pressure levels as may be selected by the user.

The method begins at the program start at step 00 in FIG. 25a. The method proceeds to step 01 in which the device is on standby. A decision block 02, typically in response to an event, the system checks if Button 1 was pressed. If Button 1 was not pressed, control returns to step 01. If Button 1 was pressed, control transfers to step 03 to begin therapy. The pump is activated, a timer is set to 0, and control is transferred to decision block 04. At decision block 04, the system checks if Button 1 was pressed. If it was not pressed, control proceeds to decision block 05. If Button 1 was pressed, control is transferred back to step 01. At decision block 05, the system is checked to determine if a partial vacuum of 113 mbar has been reached. If a partial vacuum of 113 mbar has not been reached, control transfers to decision block 06, which checks if a 5 second timer has timed out. If the partial vacuum of 113 mbar has been reached, control transfers to step 11 in which normal operation is indicated via a blinking green light. At this point, the pump may be turned off. From step 11, control transfers to decision block 12, which checks the partial vacuum to determine if the partial vacuum still exceeds 106 mbar. If partial vacuum exceeds 106 mbar, control transfers to decision block 13, which checks for Button 1. If Button 1 has been pressed, control returns to step 01. If Button 1 has not been pressed, control is transferred to step 11 to continue normal operation. At decision block 12, if the partial vacuum does not exceed 106 mbar, control is transferred to step 03, which activates the pump.

At decision block 06, if the five second timer has timed out, a counter is incremented at step 07. If the five second timer has not timed out at decision block 06, control is transferred to step 03 to continue activation of the pump. After step 07, the timer is checked to see if a second attempt to reach 113 mbar has been reached at decision block 08. If the second attempt has not been made, control returns to step 03 to continue activation of the pump. If the second attempt has been reached, an error is displayed at step 09 with a blinking red LED. At decision block 10, the system checks if button 1 has been pressed and, if so, transfers control back up to step 01. If button 1 has not been pressed, the error indication continues at step 09.

FIG. 25b also depicts a flowchart of a subroutine for checking the battery level. The subroutine begins at step 20. At decision block 21, the operating voltage is checked. If the operating voltage is less than 5 V., a battery display blinks at step 22 and then ends at step 23. If the operating voltage is not under 5 V., the subroutine ends at step 23.

It will be understood that the foregoing description of numerous implementations has been presented for purposes of illustration and description. It is not exhaustive and does not limit the claimed inventions to the precise forms disclosed. Modifications and variations are possible in light of the above description or may be acquired from practicing the invention. The claims and their equivalents define the scope of the invention.

### LIST OF REFERENCE SYMBOLS

1 Wound covering element

2 Wound cavity

3 Flat adapter

4 Wound contour

5 Sheath

6 Printed circuit

7 Absorption body

9 Wound base

10 Flat housing

11 Micropump

12 Lithium ion battery

13 Sheath

13.1 Sheath segment

14 Adhesive surface

15 Protective film element

16 Suction opening

17 Mini-USB connector sleeve

18 Mini-USB connector

19 Cable

20 Absorption body

21 Seat

22 Absorption body

22.1 Absorption body

23 Spacer body

24 Superabsorbing substance

25 Marking

26 Adhesive surface

27 Protective film element

28 Storage battery

29 Check valve

30 Segment (textile)

31 Magnetic powder layer

32 Magnetic film

33 Wound spacing grid

34 Cable

35 Button cell

36 Control module

37 Battery compartment

38 Compensation segment

39 Wound treatment area

40 Battery area 40

41, 42 Adhesive surface

43 Contour

44 Flat adapter

45 Cellulose mat

46, 47 Textile segment

48, 49 Expansion space

50 Seam

51 Flat battery (“paper battery”)

52 Pole

53 Film segment

54 Adhesive surface

55 Surface

56 Release-adhesive area

57 Opening

58 Check valve

59 Sheath

60 Sheath segment

61 Pouch

62 Window flap

63 Periphery

64 Bottom

65 Bottom segment

66 Peripheral adhesive surface

67 Surface

68 Connection

69 Cable

70 Three-way valve

71 Vacuum line

72 Outside vacuum pump

73 Outside power source

74 Suction head

75 Three-way valve

76 Suction head

77 Line

78 Three-way valve

79 Opening

100; 200; 300 Wound care device

400; 500; 600 Wound care device

700; 800; 900 Wound care device

1000; 1100 Wound care device

1200; 1300 Wound care device

1400; 1500 Wound care device

What is claimed is:

1. A wound care device for treatment of wounds by means of subatmospheric pressure in a wound area, the wound care device comprising:
at least one wound covering element;
at least one device for generating subatmospheric pressure placed on the wound covering element; and
a sensor for detecting microbial load of the wound exudation.

2. The wound care device according to claim 1, where the sensor is a germ detector.

3. The wound care device according to claim 1, where the sensor is a biosensor or a test strip with color indicators.

4. The wound care device according to claim 1, where the sensor detects multiresistant germs.

5. The wound care device according to claim 4, where the multiresistant germs are selected from the group consisting of MRSA (Methicillin-resistant Staphylococcus aureus, ORSA (Oxacillin-resistant Staphylococcus aureus), VISA (Vancomycin-intermediate Staphylococcus aureus) or VRSA (Vancomycin-resistant Staphylococcus aureus).

6. The wound care device according to claim 3, where the biosensor is placed on the principle of the immunoassay or a biochip, or on aptamers.

7. The wound care device according to claim 6, where the biosensor is in the form of a disposable product.

8. The wound care device according to claim 3, where the biosensor is combined with an operating and/or control element.

9. The wound care device according to claim 3, where the operating and/or control element includes a memory for storing and reading out the measured levels for the microbial load.

10. The wound care device according to claim 1, further comprising at least one absorption body to absorb the wound exudations.

11. The wound care device according to claim 1, further comprising at least one reservoir for collecting wound exudations.

12. The wound care device according to claim 1, further comprising a liquid-permeable wound contact layer facing the wound.

13. The wound care device according to claim 10, further comprising a barrier disposed between the device for creating subatmospheric pressure and the absorption body for absorbing the wound secretions, where the barrier is configured to prevent liquids from flowing to the device for creating subatmospheric pressure.

14. The wound care device according to claim 1, further comprising:
a connection site for an outside power supply.

15. The wound care device according to claim 1, further comprising:
a coupling, a check valve, a three-way cock, or any combination thereof arranged between the device for creating subatmospheric pressure and the wound covering element.

16. The wound care device according to claim 10, further comprising:
a coupling, a check valve, a three-way cock, or any combination thereof arranged between the device for creating subatmospheric pressure and the absorption body for absorbing the wound exudations.

17. The wound care device according to claim 10, where the absorption body includes a non-woven containing cellulose fibers.

18. The wound care device according to claim 1, further comprising:
means of dispensing medications.

19. The wound care device according to claim 1, further comprising:
means for introducing flushing medium.

20. The method for treating a wound using a wound care device comprising at least one wound covering element and at least one device for generating subatmospheric pressure which may optionally be placed on the wound covering element, the method comprising:
generating a given partial vacuum using the device for generating subatmospheric pressure;
switching off the device for creating subatmospheric pressure; and
when a partial vacuum threshold is crossed, switching on the device for creating subatmospheric pressure to generate a given partial vacuum.
